(19) United States
(12) Patent Application Publication
(10) Pub. No.: US 2021/0290675 A1
(43) Pub. Date: Sep. 23, 2021

(54) MICROFLUIDIC METHODS FOR THE PREPARATION OF CELLS

(71) Applicants: GPB SCIENTIFIC, INC., Richmond, VA (US); THE TRUSTEES OF PRINCETON UNIVERSITY, Princeton, NJ (US); UNIVERSITY OF MARYLAND, BALTIMORE, Baltimore, MD (US)

(72) Inventors: Anthony WARD, Rancho Santa Fe, CA (US); Roberto CAMPOS-GONZALEZ, Carlsbad, CA (US); Alison SKELLEY, Riverside, CA (US); Khushroo GANDHI, Palo Alto, CA (US); Michael GRISHAM, Richmond, VA (US); Curt CIVIN, Baltimore, MD (US); James C. STURM, Princeton, NJ (US)

(73) Assignees: GPB SCIENTIFIC, INC., Richmond, VA (US); THE TRUSTEES OF PRINCETON UNIVERSITY, Princeton, NJ (US); UNIVERSITY OF MARYLAND, BALTIMORE, Baltimore, MD (US)

(21) Appl. No.: 17/258,716

(22) PCT Filed: Jul. 11, 2019
(86) PCT No.: PCT/US2019/041483

§ 371 Date: Jan. 7, 2021

Related U.S. Application Data
(60) Provisional application No. 62/697,384, filed on Jul. 12, 2018.

(51) Int. Cl. A61K 35/17; C12M 1/42; C12M 3/06; C12N 15/10

(52) U.S. Cl. CPC A61K 35/17; C12N 15/102; C12M 23/16; C12M 35/02

(57) ABSTRACT

The present invention is directed to the use of microfluidics in the preparation of genetically transformed cells and compositions for therapeutic uses.

### CROSS REFERENCE TO RELATED APPLICATIONS

The present application claims the benefit of U.S. Provisional Patent Application No. 62/697,384, filed on Jul. 12, 2018.

### FIELD OF THE INVENTION

The present invention is directed primarily to microfluidic methods in which cells are genetically transformed by electroporation.

### BACKGROUND OF THE INVENTION

Cell therapy, and especially CAR-T cell therapy, has demonstrated extraordinary efficacy in treating B-cell diseases such as B-acute lymphoid leukemia (B-ALL) and B-Cell Lymphomas. As a result, the demand for autologous therapies has increased dramatically and development efforts have broadened to focus on cancers characterized by solid tumors, such as glioblastomas (Vonderheide, et al., Immunol. Rev. 257:7-13 (2014); Fousek, et al., Clin. Cancer Res. 21:3384-3392 (2015); Wang, et al., Mol. Ther. Oncolytics 3:16015 (2016); Sadelain, et al., Nature 545:423-431 (2017)). Targeted gene editing with CRISPR/Cas-9 in focused populations of autologous cells, such as stem cells, may further fuel demand (Johnson, et al., Cancer Cell Res. 27:38-58 (2017)).

The ability to produce therapeutically active cells in a way that is effective and efficient is central to making these cells available as a cost effective treatment. In this regard, Zhang et al. recently conducted an examination of optimum conditions for transforming human T cells by electroporation (see Zhang, et al; BMC Biotechnology 18:4 (2018)). They found that activation of T cells promoted transformation and that cells stimulated for about three days appeared to have the highest electroporation efficiency. The paper also studied transfection of activated T cells at different cellular concentrations and concluded that “Generally electroporation using a higher number of cells yielded more positively transfected cells.” Although raising the amount of plasmid used during electroporation increased the percentage of cells transformed, it also tended to decrease cell viability.

Microfluidic procedures such as deterministic lateral displacement are well suited to preparing therapeutically active cells, in part, because they allow the purification and concentration of cells to occur concurrently. The present invention is concerned with methods of integrating existing microfluidic methods with the genetic transformation of cells by electroporation and doing so in a way that lends itself to automation. In the description that follows, the preferred target cells are human unless otherwise indicated.

### SUMMARY OF THE INVENTION

The present invention is directed to methods for performing electroporation while cells are being processed microfluidically. The system developed allows cells to be both purified and transformed in a single continuous procedure using one or two microfluidic devices and carrying out electroporation as the cells move through the system. In some embodiments, cell concentration is increased to a predetermined level prior to electroporation. This should improve results both with respect to the number of cells transformed and with respect to the consistency of results from one batch of cells to the next.

### Methods for Genetically Engineering Target Cells

In its first aspect, the invention is directed to a method for genetically engineering a population of target cells using a system containing microfluidic devices and a component for electroporating cells. The method begins by applying a fluid composition containing the target cells to a first microfluidic device and flowing the composition from the inlets to the outlets. The cells may either be in an electroporation buffer comprising one or more transformation agents at the start of the procedure or they may be transferred into such a composition during processing. Transformation agents may include not only nucleic acids but also other factors that promote genetic engineering such as Cas9-guide RNA complexes and/or agents affecting the conditions under which electroporation occurs, e.g. agents that affect pH or salt concentration. The target cells are genetically transformed by electroporation as they flow through the device or, more preferably, as they flow through a conduit connected to an outlet of the device. This is accomplished by generating an electric field which is oriented perpendicular to the flow direction of cells, in one or more regions along the device or the conduit. The electroporated cells are then separated to remove them from electroporation buffer and transformation agents that were not transferred into cells. This separation is carried out on a second microfluidic device which is connected to the first by the conduit. Preferably, the entire method, beginning with the application of target cells to the first microfluidic device and continuing until the time that processing to remove cells from electroporation buffer and transformation agents is complete, is carried out as a single continuous process.

The target cells are preferably stem cells or leukocytes and may be obtained as part of a crude fluid composition selected from the group consisting of: blood, a biological fluid other than blood, an apheresis sample or other product derived from blood, growth medium or cell culture medium. In one embodiment, prior to being applied to the first microfluidic device, the target cells are separated from unwanted, “contaminant,” cells or particles using deterministic lateral displacement (DLD), carriers or microbeads. In a preferred embodiment, the target cells are isolated using carriers or magnetic microbeads carrying agents, preferably antibodies that bind with specificity to the target cells. As used in this context, the word “specificity” means that at least 100 (and preferably at least 1000) target cells will be bound by carriers or microbeads relative to each non-target cell bound. Once separated, the target cells may then be further purified as they pass through the first microfluidic device and may also be transferred into the electroporation buffer containing transformation agents.

In an alternative embodiment, the target cells are obtained in a crude fluid composition that also comprises contaminant particles and/or cells that are a different size from the target cells and this composition is applied directly to the first microfluidic device, i.e., without prior purification. Preferred target cells are stem cells or leukocytes and contaminants will typically include erythrocytes and/or platelets. The target cells are separated from the contaminant particles and/or cells by performing deterministic lateral displacement (DLD).

The primary characteristics of the device on which DLD is carried out are that it has at least one channel extending from a sample inlet to at least two fluid outlets, wherein the channel is bounded by a first wall and a second wall opposite to the first wall. In the channel, there is an array of obstacles arranged in rows, each subsequent row being shifted laterally with respect to a previous row, and wherein the obstacles are disposed in a manner such that, when the crude fluid composition is fluidically passed through the channel, target cells flow to one or more product outlets where a product enriched in target cells exits the device and contaminant cells or particles that are of a different size than the target cells flow to one more waste outlets that are separate from the product outlets.

In a preferred DLD purification, the crude fluid composition is entered onto the first microfluidic device at a first inlet and an electroporation buffer comprising one or more transformation agents is entered onto the first microfluidic device at a second inlet different from the first. As target cells flow though the device, they are transferred into the electroporation buffer comprising one or more transformation agents at the same time that they are separating from the contaminant particles and/or cells of a different size. At the end of the separation, the contaminant particles or cells exit the device at a waste outlet and the target cells exit the device at a product outlet. The waste cells and particles may either be reused or discarded and target cells are passed through a conduit, during which time they are electroporated (see generally FIG. 4).

After electroporation, the target cells flow onto a second microfluidic device which, like the first device, has at least one channel extending from sample inlets to at least two fluid outlets. The channel is bounded by a first wall and a second wall opposite from the first wall. An array of obstacles is arranged in rows in the channel, each subsequent row of obstacles being shifted laterally with respect to a previous row, with the obstacles being disposed in a manner such that target cells flow to one or more product outlets where a product enriched in target cells exits the device and electroporation buffer and transformation agents that are of a different size than the target cells flow to one more separate waste outlets. DLD is performed on the second device during which the target cells are transferred from the electroporation buffer into a different aqueous buffer, growth medium or culture medium.

Particularly preferred target cells are T cells and prior to, during or after DLD separation on the first microfluidic device, and prior to electroporation, these cells are bound to an activator. Activation should preferably be continued for 1-5 days before electroporation. The activator is preferably an antibody that is unbound, bound to a carrier or bound to a magnetic microbead. Most preferably, the target cells are T cells and are activated using magnetic beads coated with anti-CD3/CD28 antibodies. Activator can be present during electroporation or removed prior to electroporation. If removed, electroporation should preferably take place within one to five days thereafter. During transformation, nucleic acids will be present and Cas9-guide RNA complexes may also be present.

Preferably the above process is carried out without a Ficoll centrifugation step prior to the applying of target cells to the first microfluidic device or before electroporation. It is also preferable that target cells not be frozen prior to applying them to the first microfluidic device or between steps in the process.

### Engineering of Target Cells at Controlled Cell Concentrations

The present invention also includes methods for genetically engineering target cells in which the concentration of the cells undergoing electroporation is controlled. The method involves obtaining a sample comprising target cells of a predetermined size and cells or particles of less than the predetermined size. The sample is applied to a first inlet on a first microfluidic device and a wash fluid is applied to a second, separate inlet also on the first microfluidic device. In some embodiments, the wash fluid may be a buffer that is suitable for electroporation, i.e. it may be an “electroporation buffer,” and contain transfection agents such as nucleic acids and/or Cas9-guide RNA sequences. Alternatively, the wash fluid may be an aqueous buffer, growth medium or cell culture medium. In general, the wash fluid will be devoid of target cells and devoid of cells or particles less than the predetermined size when it is initially introduced onto the microfluidic device.

The microfluidic device is preferably designed for separating cells and particles by deterministic lateral displacement and is of a type well known in the art. It comprises at least one channel extending from a sample inlet area to one or more fluid outlets, wherein the channel is bounded by a first wall and a second wall opposite from the first wall. There is an array of obstacles arranged in rows in the channel, each subsequent row of obstacles being shifted laterally with respect to a previous row. The obstacles are disposed in a manner such that, when a crude fluid composition is applied to an inlet of the device and fluidically passed through the channel, target cells flow to a first outlet and contaminant cells or particles that are of less than the predetermined size flow to a second outlet where they may be collected or discarded as waste.

DLD is performed by flowing the sample of cells and the wash fluid through the device and the concentration of cells in the outflow from the first outlet of the device is measured, e.g., by flow cytometry. If the concentration of cells is lower than a predetermined concentration, the outflow is recirculated so as to replace all, or at least a portion, of the wash fluid being applied to the device. When the recirculation process results in the concentration of cells at the first outlet reaching the predetermined concentration, the outflow from the first outlet (containing target cells) is directed to a conduit, where, if not already present, the cells are combined with any components needed for electroporation, including transformation agents to be transferred into the cells. At the time that redirection of cells to the conduit occurs, application of sample to the device may continue, recirculation may stop and wash fluid may again flow onto the device as before. Alternatively, recirculation may continue for one or more cycles after cells start flowing into the conduit, with wash fluid being reintroduced at a later time. Electroporation is performed as the cells flow through the conduit by applying an electric field perpendicular to the direction of fluid flow.

After passing through the section of the conduit where electroporation takes place, the cells continue onto a second microfluidic device, which, like the first device, is designed to separate cells by DLD and which has a similar structure. There, the target cells are separated from transformation agents in the outflow that have not been translocated into cells and the target cells are transferred into a buffer, growth medium or cell culture medium.

The predetermined concentration of cells at which cells are directed to the conduit for transformation will vary depending on the target cells and compositions being used as well as on the objectives of the party performing the procedure. For example, the predetermined concentration might be: 0.5×10⁴ cells per ml; 1.0×10⁵ cells per ml; a 1.0×10⁶ cells per ml; 1.0×10⁷ cells per ml; 1.0×10⁸ cells per ml; or 2.0×10⁸ cells per ml. Alternatively, a party carrying out the procedure may define a predetermined concentration based on the initial concentration of cells. For example, outflow might be diverted to the conduit when, relative to the concentration in the sample, cells or particles in the outflow are concentrated by a factor of at least 3, 5, or 10. When a predetermined concentration is met or exceeded, flow from the outlet is directed to the conduit where electroporation takes place.

The amount of nucleic acid to use during electroporation may be experimentally determined but, in general, it may be about 0.1 to 3.5 μg/ml.

In some embodiments, the wash fluid will be water or an aqueous buffer, but it may also include reagents that chemically react with cells, particles or other components in the wash fluid or antibodies, carriers or activators that interact specifically with target cells or target particles. In some embodiments the wash buffer may be chosen for its suitability for electroporation and contain nucleic acids to be transferred into cells (e.g., nucleic acids encoding proteins) as well as other agents that are useful in genetically engineering cells (e.g., Cas9-guide RNA complexes).

The procedure for changing from recirculating outflow from the first outlet of the first microfluidic device may be automated and redirection of flow may be accomplished by a valve that is either part of the first outlet or that the first outlet is connected to. A second valve should also be present which controls whether wash fluid or recycled material is applied to the device. These valves and others in the system may be activated by standard electronic circuitry in response to cell count measurements or other processing parameters. In addition, the target cells or target particles may be reacted with, or bound to, a carrier, antibody, fluorescent tag, activator or compound prior to, during or after being reapplied to the first microfluidic device.

In a preferred embodiment, the target cells of a predetermined size are leukocytes (most preferably T cells) and cells less than the predetermined size are platelets and/or red blood cells. The leukocytes may be, for example, in a blood sample, a biological fluid other than blood, an apheresis sample or other product derived from blood, growth medium or cell culture medium. The nucleic acids used to transform the cells may encode chimeric antigen receptors that make the cells useful in the treatment of diseases such as cancer.

### Methods for Making CAR T Cells

More specifically, the invention includes methods for preparing cells for use as CAR T cells. The first step in this method involves obtaining a sample comprising T cells of a predetermined size and cells or particles of less than the predetermined size. Preferably the T cells are derived from a patient with cancer, an autoimmune disease or an infectious disease and will, after engineering and expansion, be used to treat the same patient. Both the sample and a wash fluid are applied to a first microfluidic device at separate inlets. In some embodiments, the wash fluid may be a buffer that is suitable for electroporation, i.e. it may be an “electroporation buffer,” and contain transfection agents such as nucleic acids and/or Cas9-guide RNA sequences. Alternatively, the wash fluid may be an aqueous buffer, growth medium or cell culture medium. The wash fluid may be devoid of target cells and devoid of cells or particles less than the predetermined size when it is initially introduced onto the microfluidic device.

The microfluidic device is designed for DLD and will be structurally similar to the devices described above. After the sample is applied to the first microfluidic device, DLD is performed and will result in the T cells being deflected to a first outlet and cells or particles of less than the predetermined size flowing to a second outlet where they may be collected or discarded as waste. The concentration of cells in the outflow at the first outlet of the device is measured, e.g., by flow cytometry. As long as the concentration at the outlet is below a predetermined value, the outflow is recirculated so as to replace, all, or at least a portion, of the wash fluid being applied to the device (see generally FIGS. 3 and 4). When the recirculation process results in the concentration of cells at the first outlet reaching the predetermined concentration, the outflow from the first outlet (containing T cells) is directed to a conduit, where, if not already present, the cells are combined with any components needed for electroporation, including transformation agents to be transferred into the cells. At the time that redirection of cells to the conduit occurs, application of sample to the device may continue, recirculation may stop and wash fluid may again flow onto the device as before. Alternatively, recirculation may continue for one or more cycles after cells start flowing into the conduit, with wash fluid being reintroduced at a later time. Electroporation is performed as the cells flow through the conduit by applying an electric field perpendicular to the direction of fluid flow.

In one preferred embodiment, the T cells are in a crude fluid composition of blood, a biological fluid other than blood, an apheresis sample or other product derived from blood, growth medium or cell culture medium and, prior to being applied to the first microfluidic device, the T cells are purified to separate them from erythrocytes, platelets and/or other cells or particles that are present in the crude fluid composition. Purification may be carried out by DLD or by using microbeads, particularly carriers or magnetic microbeads carrying agents, such as antibodies, that bind with specificity to T cells.

Before, during and/or after separation, the T cells are activated by being bound to an activator. Preferably the T cells will have been activated for a period of 1-5 days before being applied to the first microfluidic device as described above and activation will be due to the binding of magnetic beads coated with anti-CD3/CD28 antibodies. Activators may be present during DLD procedures and during electroporation. Alternatively, the activators can be removed prior to electroporation. In cases where activators are removed, electroporation should generally be performed within about 1 to 5 days thereafter.

After electroporation, T cells in the conduit flow to a second microfluidic device which, preferably, comprises an array of obstacles arranged in rows, with each subsequent row of obstacles shifted laterally with respect to a previous row. The obstacles are positioned so as to differentially deflect T cells to a first outlet and particles less than the predetermined size to a second outlet where they may be collected or discarded as waste. DLD is performed on the second device resulting in T cells flowing toward the first outlet and being transferred into buffer, growth medium or culture medium. Electroporation buffer and transfection agents flow toward the second outlet where they are collected or discarded.

The predetermined concentration of cells at which cells are directed to the conduit for transformation may be, for example, 0.5×10⁴ cells per ml; 1.0×10⁵ cells per ml; 1.0×10⁶ cells per ml; 1.0×10⁷ cells per ml; 1.0×10⁸ cells per ml; or 2.0×10⁸ cells per ml. When one of these predetermined concentrations is met or exceeded, flow from the outlet will be directed to the conduit where electroporation takes place. Alternatively, a party caring out the procedure may define a predetermined concentration based on the initial concentration of cells. For example, outflow be diverted to the conduit when relative to the concentration in the sample, cells or particles in the outflow are concentrated by a factor of at least 3, 5, or 10.

In order to facilitate the change of outflow direction at the first outlet from a recirculation circuit to the conduit for electrophoresis, a valve may be present as part of the outlet or the outlet may be connected to such a valve. A second valve should also be present which controls whether wash fluid or recycled material is applied to the device. As discussed above, the positions of these valves, and others in the system, may be electronically controlled in response to cell counts or other processing parameters.

The process for producing CAR T cells described above preferably does not include a centrifugation step prior to electrophoresis. The chimeric receptor expressed on the engineered cell may comprise: a) an extracellular region comprising antigen binding domain; b) a transmembrane region; c) an intracellular region and may optionally comprise one or more recombinant sequences that provide the cells with a molecular switch that, when triggered, reduce CAR T cell number or activity.

Once produced, the CAR T cells may be expanded in number by growing the cells in vitro. Activators or other factors may be added during this process to promote growth, with IL-2 and IL-15 being among the agents that may be used.

### BRIEF DESCRIPTION OF THE DRAWINGS

FIGS. 1A-1G: FIGS. 1A-1C illustrate different operating modes of one type of a DLD device. This includes: i) Separation (FIG. 1A), ii) Buffer Exchange (FIG. 1B) and iii) Concentration (FIG. 1C). In each mode, essentially all particles above a critical diameter are deflected in the direction of the array from the point of entry, resulting in size selection, buffer exchange or concentration as a function of the geometry of the device. In all cases, particles below the critical diameter pass directly through the device under laminar flow conditions and subsequently off the device at an outlet. DLD devices have been described in the literature along with methods of making and using the devices, see e.g., US 2016/0139012; US 2017/0333900; US 2016/0047735; US 2017/0209864; US 2017/0248508; and US 2019/0071639, each of which is hereby incorporated by reference herein in its entirety.

FIG. 1D shows a 14 lane DLD design used in separation mode. The full length of the depicted array and microchannel is 75 mm and the width is 40 mm, each individual lane is 1.8 mm across. FIGS. 1E-1F are enlarged views of the plastic diamond post array and consolidating collection ports for the exits. FIG. 1G depicts a photo of a leukapheresis product being processed using a device.

FIG. 2: FIG. 2 is a schematic showing how current individual chips have been designed to be stackable in layers to achieve throughput as demanded by any particular application.

FIG. 3: The diagram on the far left of FIG. 3 illustrates the movement of cells during DLD. Buffer and sample are applied to the device at separate inlet ports. As the sample progresses toward the outlet, cells with a size greater than the critical size of the array move from the sample stream (outer, hatched portion of the channels) to the buffer stream (center, stippled portion of the channels) and eventually exit at the product outlet. Panels 1-3 show various steps in a DLD procedure in which there is a recycling of product. In Panel 1, sample (white sample reservoir) and buffer (stippled buffer reservoir) are applied to a microfluidic device through separate inlets. Product containing cells greater than the critical size of the array is collected from the product outlet (stippled in the bottom product reservoir) and waste is collected from the waste outlet (clear in the bottom waste reservoir). Note that the valve from the product reservoir to the buffer inlet is closed whereas the valve from the buffer reservoir to the buffer inlet is open. In Panel 2, the valve from the product reservoir to the buffer inlet has been opened and the valve from the buffer reservoir to the buffer inlet has been closed. As a result product is recycled back onto the microfluidic device. In Panel 3, processing has proceeded to near completion. Total volume of waste has increased and total volume of product has decreased.

FIG. 4: FIG. 4 illustrates the basic components of a cell preparation system that uses microfluidic devices designed for DLD separations together with an integrated electroporation component. In this particular example, a sample containing cells is introduced at port F located on one side of a first microfluidic device (A) and electroporation buffer comprising one or more transformation agents is introduced from a reservoir (R), through a feed tube (S), and onto the device at port G on the other side of the device. DLD is performed by flowing the sample and buffer through the device toward outlets H and I. During this process, cells and particles larger than the critical size of the device are diverted toward outlet I, whereas particles having a size smaller than the critical size are not diverted and flow toward outlet H where they exit the device. Purified target cells exit the device in the outflow at outlet I and immediately thereafter a cell count is taken, preferably by flow cytometry. If the cell count indicates that the concentration of cells is at, or higher than a predetermined concentration, valve t directs the cells to conduit B. If the concentration is lower than the predetermined value, valve t diverts the cells to a recycle circuit (Q) where they flow to feed line (S). There, due to the actuation of valve U, the recycled material replaces the buffer from reservoir R. Once a predetermined concentration of cells is reached at outlet I, valves t and U can be repositioned so that outflow from outlet I enters into conduit B and buffer from reservoir R again enters the device at port G. During the outflow of cells into the conduit at outlet I, sample may continue being applied until it has all flowed onto the device, at which point the sample may be replaced with wash fluid that may or may not be the same as that applied to inlet G.

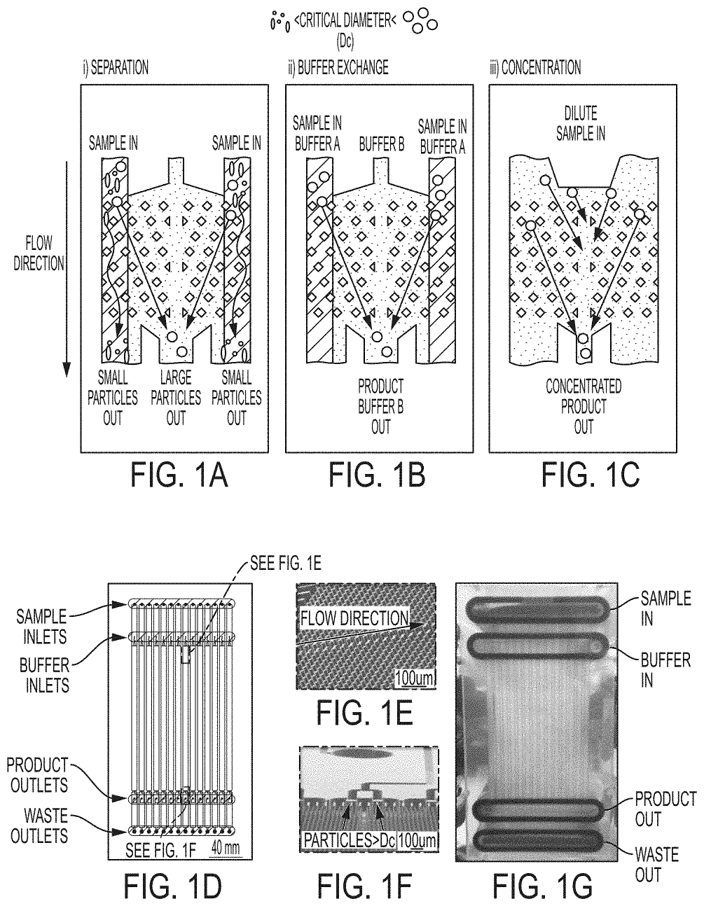

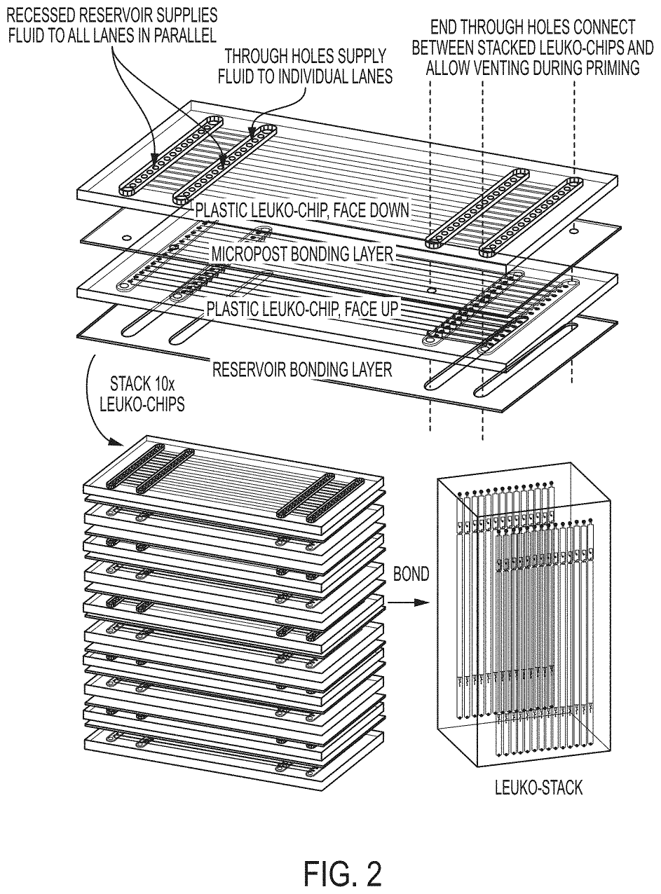

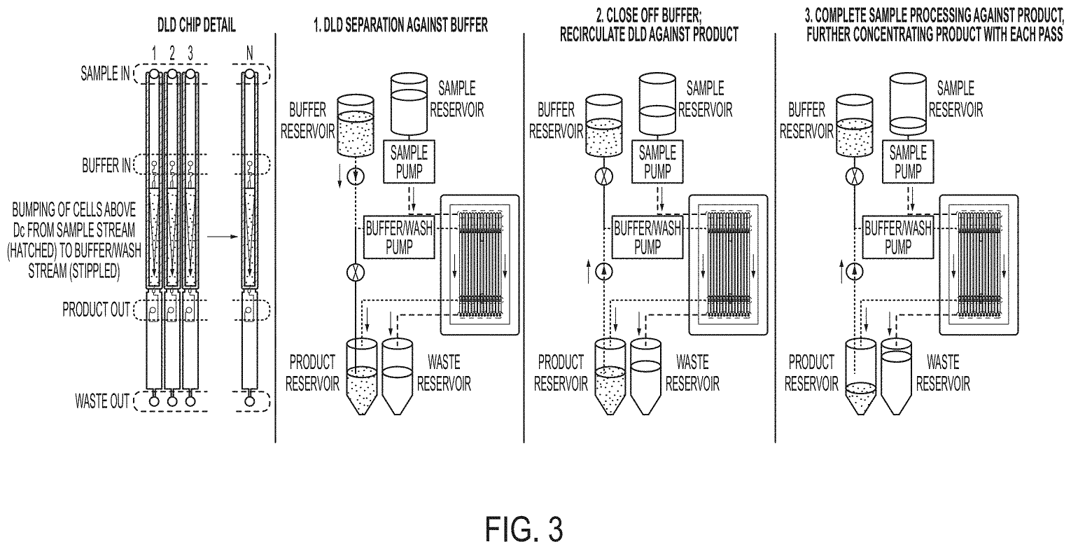

When the cells enter into the conduit, they are combined with any components needed for electroporation that are not already present, including transformation agents to be transferred into the cells. The cells then flow through electroporation section C, where they are exposed to an electric field oriented perpendicular to the direction of fluid flow.

After electroporation, the cells flow to valve K, where conduit D forms a transfection loop. If valve K and valve L are positioned so as to close off the loop, the cells will flow directly onto a second microfluidic device (E). Alternatively, the valves may be positioned so that cells flow through the transfection loop to provide additional time to complete transfection. Once on device E, the cells flow toward outlets O and P while buffer, growth medium or cell culture medium is fed onto the device through inlet port N. As a result, the cells are transferred into the new medium and exit the device at outlet P. Electroporation agents flow to outlet O and are either reused or discarded.

It is expected that the system shown would be automated so that a desired concentration of cells for electroporation could be easily chosen by someone using the system. Based on this concentration, standard circuitry and electronics may be used to activate valves in response to cell counts or other parameters.

Alternative designs for the system shown and alternative methodology should be readily apparent to those of skill in the art. For example electroporation buffer and transfection agents might not be introduced until after cells have exited the first microfluidic device and before electroporation. The sample containing T cells may either be a relatively crude (e.g., blood, biological fluid other than blood, an apheresis sample or other product derived from blood, growth medium or cell culture medium) or the T cells may have previously undergone a purification step (e.g., using commercially available magnetic beads with antibodies that specifically bind T cells and that can easily be made to release the cells after purification). Activation of T cells has been reported to greatly enhance the success of transformation by electroporation (see e.g., Zhang, et al. or Aksoy, et al., doi: http://dx.doi.org/10.1101/466243, Nov. 8, 2018). Thus, it is preferred that T cells be activated for at least 1 day and preferably 2-5 days prior to electroporation and that activated T cells be applied to the first microfluidic device and used throughout processing. For example, T cells may be separated from blood using magnetic beads coated with antibodies that are both specific for T cells and that activate the cells when bound (e.g., anti-CD3/CD28 microbeads). The cells might then be incubated with stimulator for a period of 1 to 5 days before being electroporated using the system described herein.

At the time that redirection of cells to the conduit occurs, recirculation may stop and wash fluid may again flow onto the device as before. Alternatively however, recirculation may continue for one or more cycles after cells start flowing into the conduit, with wash fluid being reintroduced at a later time.

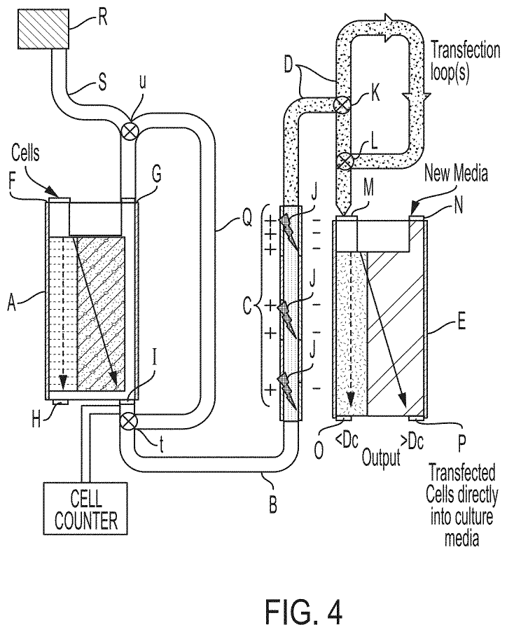

Finally, while recirculation should lead to improved electroporation efficiency and improved consistency, it is not absolutely essential to the preparation of transformed cells using the system. Thus, Applicant's system can be run without the recycle loop shown as Q in FIG. 4 and without valve t.

### DEFINITIONS

Apheresis: As used herein this term refers to a procedure in which blood from a patient or donor is separated into its components, e.g., plasma, white blood cells and red blood cells. More specific terms are “plateletpheresis” (referring to the separation of platelets) and “leukapheresis” (referring to the separation of leukocytes). In this context, the term “separation” refers to the obtaining of a product that is enriched in a particular component compared to whole blood and does not mean that absolute purity has been attained.

CAR T cells: The term “CAR” is an acronym for “chimeric antigen receptor.” A “CAR T cell” is therefore a T cell that has been genetically engineered to express a chimeric receptor.

CAR T cell therapy: This term refers to any procedure in which a disease is treated with CAR T cells. Diseases that may be treated include hematological and solid tumor cancers, autoimmune diseases and infectious diseases.

Carrier: As used herein, the term “carrier” refers an agent, e.g., a bead, or particle, made of either biological or synthetic material that is added to a preparation for the purpose of binding directly or indirectly (i.e., through one or more intermediate cells, particles or compounds) to some or all of the compounds or cells present. Carriers may be made from a variety of different materials, including DEAE-dextran, glass, polystyrene plastic, acrylamide, collagen, and alginate and will typically have a size of 1-1000 82 m. They may be coated or uncoated and have surfaces that are modified to include affinity agents (e.g., antibodies, activators, haptens, aptamers, particles or other compounds) that recognize antigens or other molecules on the surface of cells. The carriers may also be magnetized and this may provide a means of purification.

Carriers that bind “in a way that promotes DLD separation”: This term, refers to carriers and methods of binding carriers that affect the way that, depending on context, a cell, protein or particle behaves during DLD. Specifically, “binding in a way that promotes DLD separation” means that: a) the binding must exhibit specificity for a particular target cell type, protein or particle; and b) must result in a complex that provides for an increase in size of the complex relative to the unbound cell, protein or particle. In the case of binding to a target cell, there must be an increase of at least 2 μm (and alternatively at least 20, 50, 100, 200, 500 or 1000% when expressed as a percentage). In cases where therapeutic or other uses require that target cells, proteins or other particles be released from complexes to fulfill their intended use, then the term “in a way that promotes DLD separation” also requires that the complexes permit such release, for example by chemical or enzymatic cleavage, chemical dissolution, digestion, due to competition with other binders, or by physical shearing (e.g., using a pipette to create shear stress) and the freed target cells, proteins or other particles must maintain activity; e.g., therapeutic cells after release from a complex must still maintain the biological activities that make them therapeutically useful.

Target cells: As used herein “target cells” are the cells that various procedures described herein require or are designed to purify, collect, engineer etc. What the specific cells are will depend on the context in which the term is used. For example, if the objective of a procedure is to isolate a particular kind of stem cell, that cell would be the target cell of the procedure. Unless otherwise indicated, the cells referred to herein are all preferably human cells.

Isolate, purify: Unless otherwise indicated, these terms, as used herein, are synonymous and refer to the enrichment of a desired product relative to unwanted material. The terms do not necessarily mean that the product is completely isolated or completely pure. For example, if a starting sample had a target cell that constituted 2% of the cells in a sample, and a procedure was performed that resulted in a composition in which the target cell was 60% of the cells present, the procedure would have succeeded in isolating or purifying the target cell.

Bump Array: The terms “bump array” and “obstacle array” are used synonymously herein and describe an ordered array of obstacles that are disposed in a flow channel through which a cell or particle-bearing fluid can be passed.

Deterministic Lateral Displacement: As used herein, the term “Deterministic Lateral Displacement” or “DLD” refers to a process in which particles are deflected on a path through an array, deterministically, based on their size in relation to some of the array parameters. This process can be used to separate cells. However, it is important to recognize that DLD can also be used to concentrate cells and for buffer exchange.

Critical size: The “critical size” of particles passing through an obstacle array describes the size limit of particles that are able to follow the laminar flow of fluid. Particles larger than the critical size can be ‘bumped’ from the flow path of the fluid while particles having sizes lower than the critical size (or predetermined size) will not necessarily be so displaced.

Fluid flow: The terms “fluid flow” and “bulk fluid flow” as used herein in connection with DLD refer to the macroscopic movement of fluid in a general direction across an obstacle array. These terms do not take into account the temporary displacements of fluid streams for fluid to move around an obstacle in order for the fluid to continue to move in a general direction.

Tilt angle ε: In a bump array device, the tilt angle is the angle between the direction of bulk fluid flow and the direction defined by alignment of rows of sequential (in the direction of bulk fluid flow) obstacles in the array.

Array Direction: In a bump array device, the “array direction” is a direction defined by the alignment of rows of sequential obstacles in the array. A particle is “bumped” in a bump array if, upon passing through a gap and encountering a downstream obstacle, the particle's overall trajectory follows the array direction of the bump array (i.e., travels at the tilt angle relative to bulk fluid flow). A particle is not bumped if its overall trajectory follows the direction of bulk fluid flow under those circumstances.

### DETAILED DESCRIPTION OF THE INVENTION

The present invention is primarily concerned with electroporation methods that can be incorporated into microfluidic separation and concentration procedures, especially in the preparation of therapeutically active cells. The text below provides guidance regarding methods disclosed herein and information that may aid in the making and use of devices involved in carrying out those methods.

### I. Methods Involving Electroporation

### A. Introduction

Time in electroporation buffer post processing is a critical variable that may get in the way of successful electroporation (e.g., by affecting viability). DLD and other microfluidic procedures can be used to quickly process cells and change buffers. The procedures also allow for a relatively uniform exposure to current and transfection agents. When processing blood, an additional benefit is that DLD debulking removes any unwanted RBC's or other cells post processing which are problematic for electroporation, in addition to removing protein and adjusting the pH of the environment. This makes the idea of processing whole blood straight to electroporation a viable concept.

### B. Description

The methods described herein involve the introduction of agents into cells (including nucleic acids, Cas9-guide RNA complexes, peptides, proteins, or drugs) by electroporating the cells as they flow through a microfluidic system. In the example system shown in FIG. 4, cells are introduced onto a first microfluidic device that carries out separations based on size, preferably by DLD. The cells are in, or transferred during the process into, an electroporation buffer that includes nucleic acids, proteins or whatever other agents are to be introduced into the cells and then exposed to an electric field. Preferred cells are leukocytes, especially T cells, or stem cells. Most commonly these cells will be in blood, an apheresis sample, buffer, growth medium or culture medium.

In the example shown in FIG. 4, target cells are introduced onto a first DLD device and are diverted into an electroporation buffer containing transformation agents. As they flow through the device, they are directed to an outlet on the device that is separate from the outlet where smaller cells and particles go and, after passing through the outlet, may optionally be increased in concentration by directing the output to a recycle circuit. Either directly after flowing through the outlet, or after concentration, the target cells flow to a site where they are exposed to an electric field that extends longitudinally along the conduit. This arrangement allows both the strength of the electric field and the duration during which cells are exposed to be controlled.

After having passed through the portion of the conduit where they are electroporated, the cells in the example may flow directly onto a second microfluidic device for size based separation (again preferably by DLD) or they can be channeled by valves through a loop to provide additional time for transfection. Once loaded onto the second device, the target cells are transferred from the electroporation buffer into a pH appropriate wash buffer, growth medium or cell culture medium. The cells may, if desired, be cultured immediately after emerging from the device. The system may be automated and be part of a larger system used for processing cells, e.g., for CAR T cells or stem cells to be used therapeutically.

Relevant references include: Zhao, et al., “A Flow-Through Cell Electroporation Device for Rapidly and Efficiently Transfecting Massive Amounts of Cells in vitro and ex vivo,” Nature/Scientific Reports, Scientific Reports 6:18469, DOI: 10.1038/srep18469 (2016); Yang, et al., “Electroporation on microchips: the harmful effects of pH changes and scaling down” Scientific Reports 5:17817|DOI: 10.1038/srep17817 (2015); and Bao, et al., “Microfluidic electroporation of tumor and blood cells: observation of nucleus expansion and implications on selective analysis and purging of circulating tumor cells,” Integr Biol (Camb) 2:(2-3) (2010).

It is important to recognize that FIG. 4 just illustrates the basic components and concepts of the present invention. Many variations are possible both in design and methodology. For example, when the cell concentration in the recirculation loop reaches the desired value and the valves switch over to direct cells to the electroporation device and reintroduce buffer from reservoir R to inlet G, the electroporation device will see initially a high concentration of cells from outlet I and then a much lower concentration once the recirculation volume is replaced by the buffer. Because of this, the party operating the system may want to recirculate until a threshold concentration in the loop is reached (one that is below the desired concentration for electroporation, but which would reach the set concentration once passed through the device again vs. sample) and then recirculate while directing the outflow to the electroporator.

It should also be noted that recirculation volume can be fixed, as in the figure, or variable—in essence a reservoir with an inlet and outlet on opposite sides. A concentration of cells can be achieved by either collecting all the cells into a fixed volume, or by collecting the cells into a volume and then slowly reducing that volume through subsequent DLD passes. The latter approach may sometimes be somewhat more desirable because of the nature of the DLD procedure being performed.

### II. Designing Microfluidic Plates

Cells, particularly cells in compositions prepared by apheresis or leukapheresis, may be isolated by performing DLD using microfluidic devices that contain a channel through which fluid flows from one or more inlets at one end of the device to outlets at the opposite end. Basic principles of size based microfluidic separations and the design of obstacle arrays for separating cells have been provided elsewhere (see e.g., US 2014/0342375; US 2016/0139012; U.S. Pat. Nos. 7,318,902 and 7,150,812, which are hereby incorporated by reference in their entirety) and are also summarized in the sections below.

During DLD, a fluid sample containing cells is introduced into a device at an inlet and is carried along with fluid flowing through the device to outlets. As cells in the sample traverse the device, they encounter posts or other obstacles that have been positioned in rows and that form gaps or pores through which the cells must pass. Each successive row of obstacles is displaced relative to the preceding row so as to form an array direction that differs from the direction of fluid flow in the flow channel. The “tilt angle” defined by these two directions, together with the width of gaps between obstacles, the shape of obstacles, and the orientation of obstacles forming gaps are primary factors in determining a “critical size” for an array. Cells having a size greater than the critical size travel in the array direction, rather than in the direction of bulk fluid flow and particles having a size less than the critical size travel in the direction of bulk fluid flow. In devices used for blood, or blood-derived compositions, array characteristics may be chosen that result in white blood cells being diverted in the array direction whereas red blood cells and platelets continue in the direction of bulk fluid flow. In order to separate a chosen type of leukocyte from others having a similar size, a carrier may then be used that binds with specificity to that cell in a way that promotes DLD separation by, for example, forming a complex that is larger than uncompleted leukocytes. It may then be possible to carry out a separation on a device having a critical size smaller than the complexes but bigger than the uncompleted cells.

The obstacles used in devices may take the shape of columns or be triangular, square, rectangular, diamond shaped, trapezoidal, hexagonal or teardrop shaped. In addition, adjacent obstacles may have a geometry such that the portions of the obstacles defining the gap are either symmetrical or asymmetrical about the axis of the gap that extends in the direction of bulk fluid flow.

### III. Making and Operating Microfluidic Devices

General procedures for making and using microfluidic devices that are capable of separating cells on the basis of size are well known in the art. Such devices include those described in U.S. Pat. Nos. 5,837,115; 7,150,812; 6,685,841; 7,318,902; 7,472,794; and 7,735,652; all of which are hereby incorporated by reference in their entirety. Other references that provide guidance that may be helpful in the making and use of devices for the present invention include: U.S. Pat. Nos. 5,427,663; 7,276,170; 6,913,697; 7,988,840; 8,021,614; 8,282,799; 8,304,230; 8,579,117; US 2006/0134599; US 2007/0160503; US 20050282293; US 2006/0121624; US 2005/0266433; US 2007/0026381; US 2007/0026414; US 2007/0026417; US 2007/0026415; US 2007/0026413; US 2007/0099207; US 2007/0196820; US 2007/0059680; US 2007/0059718; US 2007/005916; US 2007/0059774; US 2007/0059781; US 2007/0059719; US 2006/0223178; US 2008/0124721; US 2008/0090239; US 2008/0113358; and WO2012094642 all of which are also incorporated by reference herein in their entirety. Of the various references describing the making and use of devices, U.S. Pat. No. 7,150,812 provides particularly good guidance and U.S. Pat. No. 7,735,652 is of particular interest with respect to microfluidic devices for separations performed on samples with cells found in blood (in this regard, see also US 2007/0160503).

A device can be made using any of the materials from which micro- and nano-scale fluid handling devices are typically fabricated, including silicon, glasses, plastics, and hybrid materials. A diverse range of thermoplastic materials suitable for microfluidic fabrication is available, offering a wide selection of mechanical and chemical properties that can be leveraged and further tailored for specific applications.

Techniques for making devices include Replica molding, Softlithography with PDMS, Thermoset polyester, Embossing, Injection Molding, Laser Ablation and combinations thereof. Further details can be found in “Disposable microfluidic devices: fabrication, function and application” by Fiorini, et al. (BioTechniques 38:429-446 (March 2005)), which is hereby incorporated by reference herein in its entirety. The book “Lab on a Chip Technology” edited by Keith E. Herold and Avraham Rasooly, Caister Academic Press Norfolk UK (2009) is another resource for methods of fabrication, and is hereby incorporated by reference in its entirety.

High-throughput embossing methods such as reel-to-reel processing of thermoplastics is an attractive method for industrial microfluidic chip production. The use of single chip hot embossing can be a cost-effective technique for realizing high-quality microfluidic devices during the prototyping stage. Methods for the replication of microscale features using thermoplastics, polymethylmethacrylate (PMMA) and/or polycarbonate (PC), are described in “Microfluidic device fabrication by thermoplastic hot-embossing” by Yang, et al. (Methods Mol. Biol. 949: 115-23 (2013)), which is hereby incorporated by reference in its entirety

The flow channel can be constructed using two or more pieces which, when assembled, form a closed cavity (preferably one having orifices for adding or withdrawing fluids) having the obstacles disposed within it. The obstacles can be fabricated on one or more pieces that are assembled to form the flow channel, or they can be fabricated in the form of an insert that is sandwiched between two or more pieces that define the boundaries of the flow channel.

The obstacles may be solid bodies that extend across the flow channel, in some cases from one face of the flow channel to an opposite face of the flow channel. Where an obstacle is integral with (or an extension of) one of the faces of the flow channel at one end of the obstacle, the other end of the obstacle can be sealed to or pressed against the opposite face of the flow channel. A small space (preferably too small to accommodate any particles of interest for an intended use) is tolerable between one end of an obstacle and a face of the flow channel, provided the space does not adversely affect the structural stability of the obstacle or the relevant flow properties of the device.

Obstacles in adjacent columns can be offset from one another by a degree characterized by a tilt angle, designated ε (epsilon). The tilt angle can be selected and the columns can be spaced apart from each other such that 1/ε (when expressed in radians) is an integer, and the columns of obstacles repeat periodically. The obstacles in a single column can also be offset from one another by the same or a different tilt angle.

Surfaces can be coated to modify their properties and polymeric materials employed to fabricate devices, can be modified in many ways. In some cases, functional groups such as amines or carboxylic acids that are either in the native polymer or added by means of wet chemistry or plasma treatment are used to crosslink proteins or other molecules. DNA can be attached to COC and PMMA substrates using surface amine groups. Surfactants such as Pluronic® can be used to make surfaces hydrophilic and protein repellant by adding Pluronic® to PDMS formulations. In some cases, a layer of PMMA is spin coated on a device, e.g., microfluidic chip and PMMA is “doped” with hydroxypropyl cellulose to vary its contact angle.

To reduce non-specific adsorption of cells or compounds, e.g., released by lysed cells or found in biological samples, onto the channel walls, one or more walls may be chemically modified to be non-adherent or repulsive. The walls may be coated with a thin film coating (e.g., a monolayer) of commercial non-stick reagents, such as those used to form hydrogels. Additional examples of chemical species that may be used to modify the channel walls include oligoethylene glycols, fluorinated polymers, organosilanes, thiols, poly-ethylene glycol, hyaluronic acid, bovine serum albumin, poly-vinyl alcohol, mucin, poly-HEMA, methacrylated PEG, and agarose. Charged polymers may also be employed to repel oppositely charged species. The type of chemical species used for repulsion and the method of attachment to the channel walls can depend on the nature of the species being repelled and the nature of the walls and the species being attached. Such surface modification techniques are well known in the art. The walls may be functionalized before or after the device is assembled.

### IV. CAR T Cells

Methods for making and using CAR T cells are well known in the art. Procedures have been described in, for example, U.S. Pat. Nos. 9,629,877; 9,328,156; 8,906,682; US 2017/0224789; US 2017/0166866; US 2017/0137515; US 2016/0361360; US 2016/0081314; US 2015/0299317; and US 2015/0024482; each of which is incorporated by reference herein in its entirety.

### V. Separation Processes that Use DLD

DLD devices can be used to purify cells, cellular fragments, cell adducts, or nucleic acids. These devices can also be used to separate a cell population of interest from a plurality of other cells. Separation and purification of blood components using devices can be found, for example, in US Publication No. US2016/0139012, the teaching of which is incorporated by reference herein in its entirety.

### VI. Technological Background

Without being held to any particular theory, a general discussion of some technical aspects of microfluidics may help in understanding factors that affect separations carried out in this field. A variety of microfabricated sieving matrices have been disclosed for separating particles (Chou, et. al., Proc. Natl. Acad. Sci. 96:13762 (1999); Han, et al., Science 288:1026 (2000); Huang, et al., Nat. Biotechnol. 20:1048 (2002); Turner et al., Phys. Rev. Lett. 88(12):128103 (2002); Huang, et al., Phys. Rev. Lett. 89:178301 (2002); U.S. Pat. Nos. 5,427,663; 7,150,812; 6,881,317). Bump array (also known as “obstacle array”) devices have been described, and their basic operation is explained, for example in U.S. Pat. No. 7,150,812, which is incorporated herein by reference in its entirety. A bump array operates essentially by segregating particles passing through an array (generally, a periodically-ordered array) of obstacles, with segregation occurring between particles that follow an “array direction” that is offset from the direction of bulk fluid flow or from the direction of an applied field (U.S. Pat. No. 7,150,812).

### A. Bump Arrays

In some arrays, the geometry of adjacent obstacles is such that the portions of the obstacles defining the gap are symmetrical about the axis of the gap that extends in the direction of bulk fluid flow. The velocity or volumetric profile of fluid flow through such gaps is approximately parabolic across the gap, with fluid velocity and flux being zero at the surface of each obstacle defining the gap (assuming no-slip flow conditions) and reaching a maximum value at the center point of the gap. The profile being parabolic, a fluid layer of a given width adjacent to one of the obstacles defining the gap contains an equal proportion of fluid flux as a fluid layer of the same width adjacent to the other obstacle that defines the gap, meaning that the critical size of particles that are ‘bumped’ during passage through the gap is equal regardless of which obstacle the particle travels near.

In some cases, particle size-segregating performance of an obstacle array can be improved by shaping and disposing the obstacles such that the portions of adjacent obstacles that deflect fluid flow into a gap between obstacles are not symmetrical about the axis of the gap that extends in the direction of bulk fluid flow. Such lack of flow symmetry into the gap can lead to a non-symmetrical fluid flow profile within the gap. Concentration of fluid flow toward one side of a gap (i.e., a consequence of the non-symmetrical fluid flow profile through the gap) can reduce the critical size of particles that are induced to travel in the array direction, rather than in the direction of bulk fluid flow. This is because the non-symmetry of the flow profile causes differences between the width of the flow layer adjacent to one obstacle that contains a selected proportion of fluid flux through the gap and the width of the flow layer that contains the same proportion of fluid flux and that is adjacent the other obstacle that defines the gap. The different widths of the fluid layers adjacent to obstacles define a gap that exhibits two different critical particle sizes. A particle traversing the gap can be bumped (i.e., travel in the array direction, rather than the bulk fluid flow direction) if it exceeds the critical size of the fluid layer in which it is carried. Thus, it is possible for a particle traversing a gap having a non-symmetrical flow profile to be bumped if the particle travels in the fluid layer adjacent to one obstacle, but to be not-bumped if it travels in the fluid layer adjacent to the other obstacle defining the gap.

In another aspect, decreasing the roundness of edges of obstacles that define gaps can improve the particle size-segregating performance of an obstacle array. By way of example, arrays of obstacles having a triangular cross-section with sharp vertices can exhibit a lower critical particle size than do arrays of identically-sized and -spaced triangular obstacles having rounded vertices.

Thus, by sharpening the edges of obstacles defining gaps in an obstacle array, the critical size of particles deflected in the array direction under the influence of bulk fluid flow can be decreased without necessarily reducing the size of the obstacles. Conversely, obstacles having sharper edges can be spaced farther apart than, but still yield particle segregation properties equivalent to, identically-sized obstacles having less sharp edges.

### B. Fractionation Range

Objects separated by size on microfluidic include cells, biomolecules, inorganic beads, and other objects. Typical sizes fractionated range from 100 nanometers to 50 micrometers. However, larger and smaller particles may also sometimes be fractionated.

### C. Volumes

Depending on design, a device or combination of devices might be used to process between about 10 μl to at least 500 μl of sample, between about 500 μl and about 40 mL of sample, between about 500 μl and about 20 mL of sample, between about 20 mL of sample and about 200 mL of sample, between about 40 mL of sample and about 200 mL of sample, or at least 200 mL of sample.

### D. Channels

A device can comprise one or multiple channels with one or more inlets and one or more outlets. Inlets may be used for sample or crude (i.e., unpurified) fluid compositions, for buffers or to introduce reagents. Outlets may be used for collecting product or may be used as an outlet for waste. Channels may be about 0.5 to 100 mm in width and about 2-200 mm long but different widths and lengths are also possible. Depth may be 1-1000 μm and there may be anywhere from 1 to 100 channels or more present. Volumes may vary over a very wide range from a few μl to many ml and devices may have a plurality of zones (stages, or sections) with different configurations of obstacles.

### E. Stackable Chips

A device can include a plurality of stackable chips. A device can comprise about 1-50 chips. In some instances, a device may have a plurality of chips placed in series or in parallel or both.

All references cited herein are fully incorporated by reference. Having now fully described the invention, it will be understood by one of skill in the art that the invention may be performed within a wide and equivalent range of conditions, parameters and the like, without affecting the spirit or scope of the invention or any embodiment thereof.

1-17. (canceled)

18. A method for genetically engineering a population of target cells of a predetermined size, comprising:
a) obtaining a sample comprising target cells of a predetermined size and cells or particles of less than the predetermined size;
b) applying the sample to a first inlet on a first microfluidic device and applying a wash fluid to a second, separate inlet on the first microfluidic device, wherein the microfluidic device comprises an array of obstacles arranged in rows, with each subsequent row of obstacles shifted laterally with respect to a previous row, and wherein the obstacles are positioned so as to differentially deflect target cells to a first outlet and to direct the cells or particles of less than the predetermined size to a second outlet where they may be collected or discarded as waste;
c) performing deterministic lateral displacement (DLD) by flowing the sample and wash fluid through the device;
d) measuring the concentration of cells in outflow at the first outlet of the device and recirculating the outflow so as to replace, all, or at least a portion, of the wash fluid being applied to the device, and continuing the recirculation process until the concentration of cells reaches a predetermined concentration;
e) when the predetermined concentration is reached in step c), directing the outflow from the first outlet to a conduit, where it is combined with one or more transformation agents to be transferred into the cells and performing electroporation as the cells flow through the conduit;
f) flowing the electroporated cells from step d) through the conduit and onto a second device that separates the target cells from transformation agents in the outflow from the conduit and transfers the target cells into a stabilization buffer or growth medium. cm 19. (canceled)

20. The method of claim 18, wherein the wash fluid is either electroporation buffer comprising one or more transformation agents, an aqueous buffer, growth medium or cell culture medium.

21. (canceled)

22. The method of claim 18, wherein recirculation of the outflow from the first outlet of the first device continues until the cell concentration reaches at least 1.0×10⁶ cells per ml.

23-25. (canceled)

26. The method of claim 18, wherein recirculation is continued until, relative to the concentration in the sample, cells or particles are concentrated by a factor of at least 3.

27-32. (canceled)

33. The method of claim 18, wherein the target cells are leukocytes and cells less than the predetermined size are platelets or red blood cells.

34. The method of claim 33, wherein the sample is blood or a composition that has been obtained by performing apheresis or leukapheresis on blood.

35. (canceled)

36. The method of claim 18, wherein the target cells are T cells and, prior to electroporation, the T cells are activated.

37. The method of claim 18, wherein the target cells are T cells and, wherein:
i) in step a), the T cells are obtained in a crude fluid composition of blood, a biological fluid other than blood, an apheresis sample or other product derived from blood, growth medium or cell culture medium; and
ii) prior to being applied to the first microfluidic device, the T cells are purified to separate the T cells from erythrocytes, platelets and/or other cells or particles that are present in a crude fluid composition; and
iii) before, during and/or after the separation of step ii) the T cells are activated by being bound to an activator.

38. (canceled)

39. The method of claim 37, wherein, in step ii), T cells are purified using magnetic microbeads carrying agents that bind with specificity to T cells.

40-41. (canceled)

42. The method of claim 37, wherein said method is used in a process for producing CAR-T cells.

43. (canceled)

44. A method for preparing cells for use as CAR T cells, comprising:
a) obtaining a sample comprising T cells of a predetermined size, and cells or particles of less than the predetermined size;
b) applying both the sample and a wash fluid to a first microfluidic device at separate inlets, wherein the microfluidic device comprises an array of obstacles arranged in rows, with each subsequent row of obstacles shifted laterally with respect to a previous row, and wherein the obstacles are positioned so as to differentially deflect T cells to a first outlet and to direct the cells or particles of less than the predetermined size to a second outlet where they may be collected or discarded as waste;
c) performing deterministic lateral displacement (DLD) by flowing the sample and wash fluid through the device;
d) measuring the concentration of cells in the outflow at the first outlet of the device and recirculating the outflow so as to replace, all, or at least a portion, of the wash fluid being applied to the device and continuing the recirculation process until the concentration of cells reaches a predetermined concentration;
e) when the predetermined concentration is reached in step c), directing the outflow from the first outlet to a conduit, where it is combined with one or more transformation agents to be transferred into the cells and performing electroporation as the cells flow through the conduit, wherein said transformation agents comprise nucleic acids used to produce chimeric antigen receptors;
feeding the electroporated cells from step e) through the conduit and onto a second device that separates the target cells from transformation agents in the outflow from the conduit and transfers the target cells into a stabilization buffer or growth medium.

45. The method of claim 44, wherein:
i) in step a), the T cells are obtained in a crude fluid composition of blood, a biological fluid other than blood, an apheresis sample or other product derived from blood, growth medium or cell culture medium; and
ii) prior to being applied to the first microfluidic device, the T cells are purified to separate the T cells from erythrocytes, platelets and/or other cells or particles that are present in the crude fluid composition; and
iii) before, during and/or after the separation of step ii) the T cells are activated by being bound to an activator.

46. (canceled)

47. The method of claim 45, wherein, in step ii), T cells are purified using magnetic microbeads carrying agents that bind with specificity to T cells.

48. The method of claim 47, wherein the agents that bind with specificity to the T cells are antibodies.

49. (canceled)

50. The method of claim 44, wherein T cells have been activated for a period of 1-5 days before being applied to the first microfluidic device in step b).

51. The method of claim 44, wherein the second microfluidic device comprises an array of obstacles arranged in rows, with each subsequent row of obstacles shifted laterally with respect to a previous row, and wherein the obstacles are positioned so as to differentially deflect target cells to a first outlet and to direct the cells or particles of less than the predetermined size to a second outlet where they may be collected or discarded as waste and wherein, in step e) target cells are purified and transferred into stabilization buffer or growth medium by DLD.

52. The method of claim 44, wherein the wash fluid is either electroporation buffer comprising one or more transformation agents, an aqueous buffer, growth medium or cell culture medium.

53. (canceled)

54. The method of claim 44, wherein recirculation of the outflow from the first outlet of the first device continues until the cell concentration reaches at least 1.0×10⁶ cells per ml.

55-57. (canceled)

58. The method of claim 44, wherein recirculation is continued until, relative to the concentration in the sample, cells or particles are concentrated by a factor of at least 3.

59-63. (canceled)

64. The method of claim 44, wherein said T cells are derived from a patient with cancer, an autoimmune disease or an infectious disease.
